(19) United States
(12) Patent Application Publication
(10) Pub. No.: US 2026/0117144 A1
(43) Pub. Date: Apr. 30, 2026

(54) METHODS FOR EXTRACTING CORN KERNEL OIL

(71) Applicant: University of Kansas, Lawrence, KS (US)

(72) Inventors: Prathusha Kothinti, Lawrence, KS (US); Alan Martin Allgeier, Lawrence, KS (US); Thomas P Binder, Marion, IA (US); Bala Subramaniam, Lawrence, KS (US)

(73) Assignee: University of Kansas, Lawrence, KS (US)

(21) Appl. No.: 19/369,744

(22) Filed: Oct. 27, 2025

Related U.S. Application Data
(60) Provisional application No. 63/712,616, filed on Oct. 28, 2024.

(51) Int. Cl. C11B 1/10; C12P 7/10

(52) U.S. Cl. CPC C11B 1/10; C12P 7/10

(57) ABSTRACT

Provided are methods for extracting corn oil from corn kernels using an extraction solvent, e.g., pressurized propane. In embodiments, such a method comprises exposing corn kernels to pressurized propane to extract corn oil from the corn kernels in a stirred tank extractor comprising an impeller, thereby providing an extraction mixture comprising extracted corn oil, pressurized propane, and processed corn kernels; and separating the extracted corn oil from the extraction mixture, thereby providing separated corn oil.

### CROSS-REFERENCE TO RELATED APPLICATIONS

The present application claims priority to U.S. provisional patent application No. 63/712,616 that was filed Oct. 28, 2024, the entire contents of which are incorporated herein by reference.

### BACKGROUND

Ethanol production from corn has evolved over decades to improve efficiency and generation of valuable coproducts. Corn kernels contain around 5% of corn oil, which is recognized as a food source. Corn oil, rich in fatty acids, contains high levels of polyunsaturated fatty acids, making it valuable for human diets while reducing the risk of heart diseases. However, corn oil for human consumption is not currently generated from ethanol manufacturing processes (dry mills). Instead, the corn oil is extracted as a terminal unit operation in the plant for either animal nutrition, as a component of the distiller's dried grain and solubles (DDGS), or conversion to biodiesel.

### SUMMARY

Provided are methods for extracting corn oil from corn kernels before saccharification and fermentation using an extraction solvent, e.g., pressurized propane. The methods may be carried out in a dry grind corn mill (an ethanol plant) with reduced energy consumption for removal of the extraction solvent.

The present methods are illustrated in the Examples, below, in which ground corn kernels were treated in a pressure vessel with pressurized propane under conditions sufficient to extract corn oil from the ground corn kernels into the pressurized propane. A liquid oil-propane mixture was transferred from the pressure vessel to a separation vessel in which propane was evaporated. The product stream from the separation vessel was corn oil, free of propane and comprising a substantial amount of the corn oil originally present in the corn kernels. The specific recovery of corn oil from the corn kernels may be tuned by the selection of operating parameters of the present methods. For comparison to propane, extractions with hexane and ethanol were also conducted in the Examples, below.

In embodiments, a method for processing corn kernels comprises exposing corn kernels to pressurized propane to extract corn oil from the corn kernels in a stirred tank extractor comprising an impeller, thereby providing an extraction mixture comprising extracted corn oil, pressurized propane, and processed corn kernels; and separating the extracted corn oil from the extraction mixture, thereby providing separated corn oil.

Other principal features and advantages of the disclosure will become apparent to those skilled in the art upon review of the following drawings, the detailed description, and the appended claims.

### BRIEF DESCRIPTION OF THE DRAWINGS

Illustrative embodiments of the disclosure will hereafter be described with reference to the accompanying drawings.

FIG. 1 shows a schematic of a method for extracting corn oil from corn kernels according to an illustrative embodiment (dashed box 100). The illustrative method may be carried out in an ethanol plant as an additional step in the production of ethanol illustrated in box 102.

FIG. 2 shows a schematic diagram of a system and method for extracting corn oil from corn kernels according to an illustrative embodiment (a) using pressurized propane. Also shown are comparative systems and methods (b) using ethanol and hexane.

FIG. 3 shows yield of extracted corn oil from ground corn kernels using ethanol, hexane, and pressurized propane as solvents at different solvent-to-solid ratios. Extractions were conducted at 328.15 K for 90 min, under atmospheric pressure for ethanol and hexane, and 3.6 MPa for propane (containing 2 MPa of nitrogen). Error bars represent one standard deviation from triplicate runs.

FIG. 4 shows tocopherols fraction percentage (Vitamin E) for commercial corn oil (CCO), propane extracted corn oil (PCO), and ethanol extracted corn oil (ECO).

FIG. 5 shows ethanol yields (v/w %) for commercial corn (no prior oil extraction, CC), propane-extracted corn (PEC), hexane-extracted corn (HEC), and ethanol-extracted corn (EEC).

### DETAILED DESCRIPTION

Provided are methods for extracting corn oil from corn kernels. In embodiments, such a method comprises exposing corn kernels to an extraction solvent under conditions to extract corn oil from the corn kernels into the extraction solvent. The exposure step provides an extraction mixture comprising (or consisting of) extracted corn oil, the extraction solvent, and processed corn kernels. The methods further comprise separating the extracted corn oil from the extraction mixture, e.g., by evaporating the extraction solvent. This may be accomplished by exposing the extraction mixture to conditions to vaporize the extraction solvent, thereby providing separated corn oil. The processed corn kernels may be filtered out of the extraction mixture prior to separating the extracted corn oil. Vaporized extraction solvent may be liquified and reused (i.e., recycled) to carry out the exposure step one or more additional times, e.g., with one or more fresh batches of corn kernels. Thus, the steps of the present methods may form a closed loop process that may be carried out continuously with minimal external inputs.

The corn kernels used in the present methods refer to the fruit of a corn plant. The corn kernels comprise corn germ as well other the components of the fruit. Thus, corn kernels are a composite material distinct from the individual components of corn kernels, including corn germ. The corn kernels used in the present methods may be ground into corn kernel particles having a smaller diameter (e.g., less than 5 mm, less than 2 mm, less than 1 mm, in a range of from 0.1 mm to 1.5 mm) as compared to unground corn kernels. However, the corn kernels are generally not subjected to a process such as wet corn milling which may separate individual components from one another. The corn kernels are also distinguished from distillers dried grains and distillers dried grains with solubles which are products resulting from processing of corn in an ethanol plant.

The extraction solvent used in the present methods comprises a hydrocarbon compound in its liquid phase. Light alkanes may be used as the hydrocarbon. However, the extraction solvent is not hexane or ethanol. Pressurized propane is a useful extraction solvent. Pressurized propane refers to the use of temperatures and pressures during the exposure step that ensure that the propane is in its liquid phase, although an amount of propane vapor may be present. Thus, “pressurized propane” encompasses liquid propane and any propane vapor that may also be present. The present methods do not require the use of any co-solvents. In embodiments, the extraction solvent consists of pressurized propane. In embodiments, the extraction mixture consists of extracted corn oil, pressurized propane, processed corn kernels, and optionally, water. The propane used to provide the pressurized propane need not have the highest purity. In embodiments, the propane is 98% pure.

The exposure step may be carried out in a reactor configured to contain the extraction mixture under pressure with mixing (such a reactor may be referred to as a pressure vessel, reactor vessel, extraction vessel, or extractor). In embodiments, the reactor configured to contain the extraction mixture under pressure with mixing is a stirred tank extractor comprising an impeller. It has been found that use of the impeller enhances the rate of extraction of corn oil from the corn kernels. The stirred tank extractor may be a continuous stirred tank extractor. The continuous stirred tank extractor is configured to receive and deliver flowing input (e.g., extraction solvent, corn kernels) streams and flowing output streams (e.g., liquid phase portion of extraction mixture). In other embodiments, the exposure step is carried out in a solid-liquid extraction column.

The conditions used during the exposure step include parameters such as an extraction pressure, extraction temperature, extraction time, ratios of extraction solvent to corn kernels, and mixing rate. As noted above, the extraction pressure and extraction temperature are those at which the extraction solvent is in its liquid phase (although some vapor phase extraction solvent may be present). Otherwise, these parameters (as well as the others listed above) may be selected to achieve a desired result, e.g., a maximum yield of corn oil. Illustrative values of these parameters include the following: extraction pressures may be in a range of from 7 to 150 bar (including from 7 to 100 bar, from 7 to 75 bar, from 7 to 40 bar, from 10 to 25 bar, from 20 bar to 40 bar, and ranges between any of these individual values); extraction temperatures may be in a range of from 20 to 100° C. (including from 45 to 70° C., from 50 to 60° C., and ranges between any of these individual values); extraction times may be in a range of from 15 to 360 min (including from 30 to 120 min, from 60 min to 100 min, and ranges between any of these individual values); solvent-to-solid ratios ((volume of extraction solvent):(mass of corn kernels)) may be in a range of from 10:1 to 1:1 (including from 6:1 to 2:1, from 5:1 to 3:1, and ranges between any of these individual values); and mixing rate may be in a range of from 10 rpm to 1000 rpm (including from 250 rpm to 500 rpm). Regarding extraction pressure, an inert gas (e.g., N₂) may be included in the reactor and the extraction pressure refers to a total pressure in the reactor, which as described above, may include an amount of vaporized extraction solvent.

The separation step may be carried out in a separator configured to contain the liquid phase portion of the extraction mixture and evaporate the extraction solvent (such a separator may be referred to as a flash tank or a flash separator) so as to provide a stream of vaporized extraction solvent (e.g., a vapor propane stream). The conditions used during the separation step include parameters such as a separation temperature and a separation pressure. These parameters may be selected as noted above and illustrative values include the following: separation temperature in a range of from −30 to 55° C. and separation pressure from 1 to 70 bar.

The system for carrying out the present methods may comprise any of the reactors and separators described above. The system may be configured with heat integration, wherein heat is transferred to specific input/output streams and/or system components, e.g., to heat corn kernels before and/or during the exposure step. This includes heating to achieve the extraction temperature.

The present methods provide high quality separated corn oil at high yields. Regarding quality, the separated corn oil provided by the present methods is suitable for human consumption. By contrast to hexane, extraction solvents such as pressurized propane are FDA-approved food grade solvents. In addition, by contrast to conventional methods for extracting corn oil, the amount of extraction solvent (e.g., pressurized propane) and other components (e.g., phospholipids, color-formers, odor-formers, etc.) in the separated corn oil provided by the present methods is less.

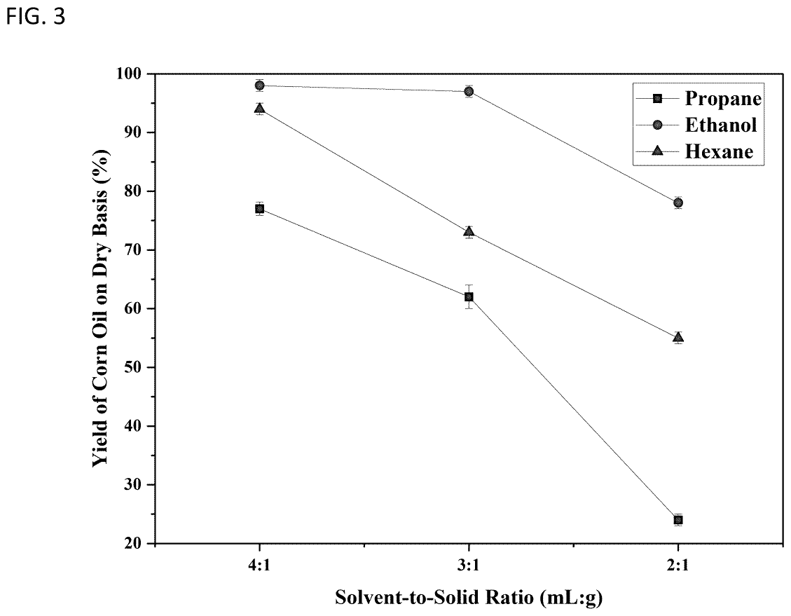

Regarding corn oil yield, this may be measured as described in the Example 3 below (Equation 6). (See also FIG. 3.) In embodiments, the yield is at least 70%, at least 72%, at least 74%, at least 76%, at least 78%, at least 80%, at least 85%, at least 90%, at least 95%, 100%, or a range between any of these values. Thus, up to all of the corn oil present in the corn kernels may be extracted and separated by the present methods.

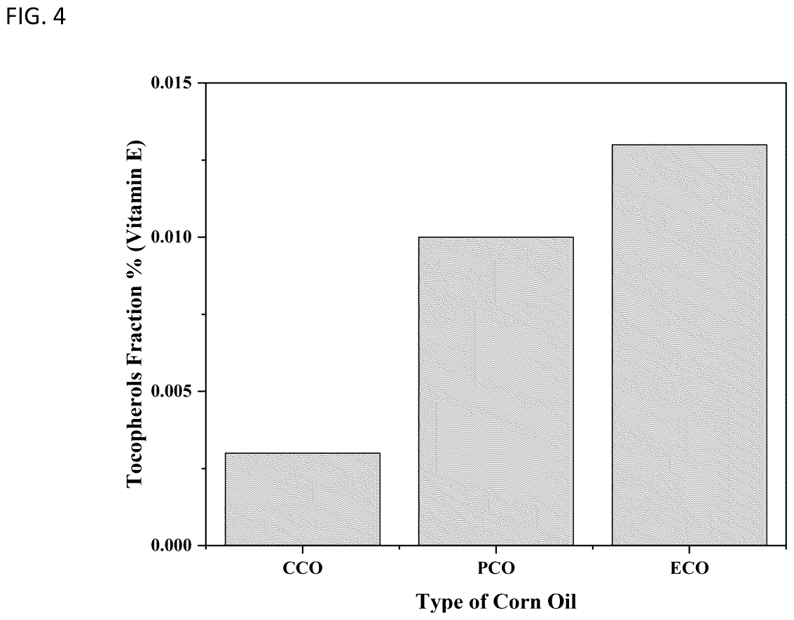

The separated corn oil may be used in a variety of applications including food products for humans and animals; a raw material for obtaining fat through various lipid modifications; a carrier for pharmaceutical formulations; production of ethanol. Examples 2 and 3, below, also show that a substantial amount of vitamins (e.g., tocopherols) may be recovered from the separated corn oil (see FIG. 4). The tocopherol fraction in the separated corn oil may be at least 0.0075%, at least 0.010%, or at least 0.0125%, or a range of between any of these values. Tocopherol fraction may be measured as described in Example 3, below.

The methods may further comprise recovering the processed corn kernels from the extraction mixture. Recovered processed corn kernels may be subjected to additional steps as desired, including any of the steps carried out in an ethanol plant using corn, e.g., fermentation, saccharification, etc. Thus, the present methods may be carried out in conjunction with any of the steps carried out in an ethanol plant, including prior to a fermentation step in such a plant. This is further described below.

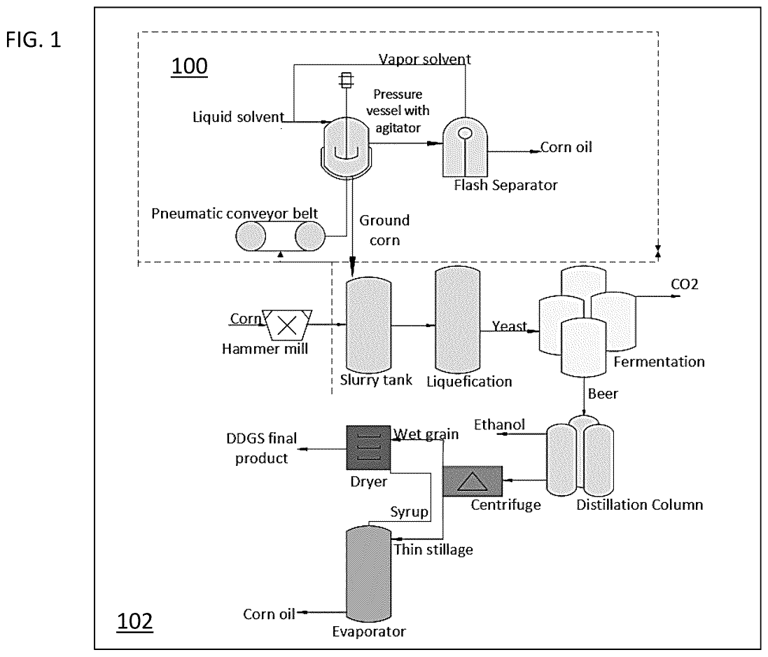

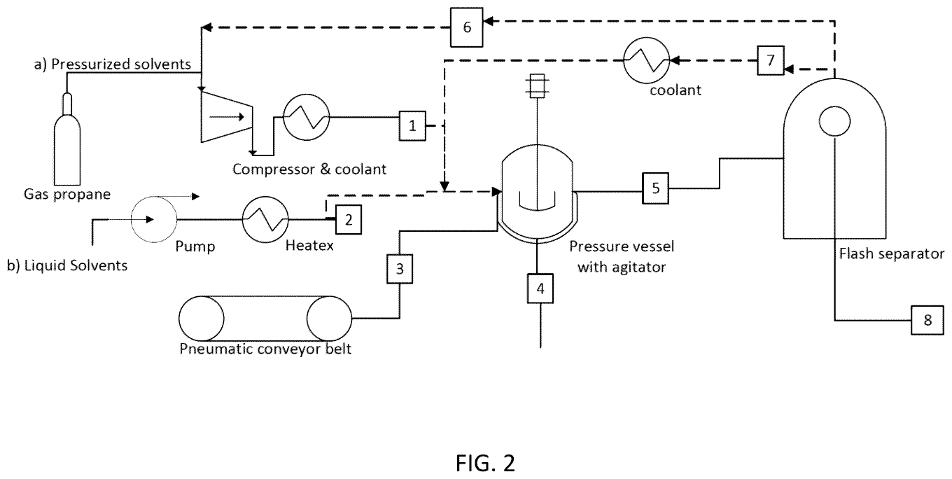

An illustrative system that may be used to carry out the present methods is shown in box 100 of FIG. 1. (See also the “a” portion of FIG. 2.) The illustrative system includes a pneumatic conveyor belt for providing corn kernels to a pressure vessel with agitator in which the exposure step of the present methods is carried out. The extraction solvent (e.g., pressurized propane) is also provided to the pressure vessel. After the exposure step, the liquid phase portion of the extraction mixture is fed to a flash separator in which the separation step of the present methods is carried out to provide the separated corn oil. The vaporized extraction solvent from the flash separator may be liquified and fed back to the pressure vessel. The liquification may be carried out by including a compressor in the system. Heat released during compression may be transferred to specific input/output streams and/or system components as described above, e.g., to heat corn kernels before and/or during the exposure step. Processed corn kernels may be recovered from the pressure vessel as described above.

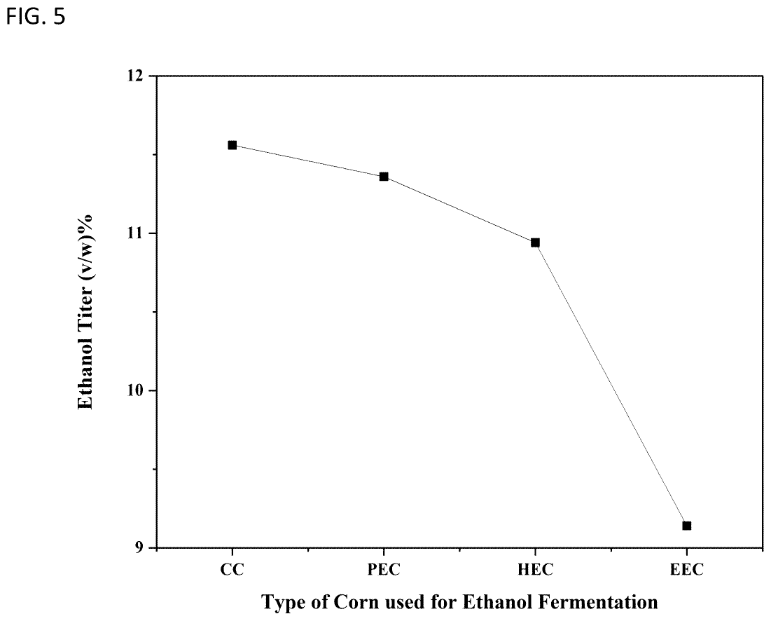

Box 102 of FIG. 1 further includes various steps that may be carried out in an ethanol plant, illustrating that the present methods may be carried out in conjunction with such steps. For example, the corn kernels to be provided to the pressure vessel may be ground corn kernels derived from a hammer milling step being carried out in the ethanol plant. The processed corn kernels from the pressure vessel may be used as a feedstock in other steps being carried out in the ethanol plant, including fermentation. This is illustrated in Examples 2 and 3, below. These Examples also show that fermentation of the processed corn kernels results in high ethanol titer values (see FIG. 5). The ethanol titer may be at least 11 (v/w) %, at least 11.4 (v/w) %, at least 11.6 (v/w) %, at least 11.8 (v/w) %, at least 12 (v/w) % or a range between any of these values. Ethanol titer may be measured as described in Example 3, below.

### EXAMPLES

### Example 1

### INTRODUCTION

This Example presents the experimental results of corn oil extraction from ground corn using pressurized propane as an extraction solvent. For comparison, experiments using ethanol and hexane were also conducted.

### Materials

Ground corn kernels were obtained from Trenton Agri Products LLC, Kansas, USA. Ethyl Alcohol (pure, 200 proof, anhydrous, >99.5), n-Hexane (99%), and propane (98%) gas cylinders were purchased from Sigma Aldrich, USA.

### Extraction of Corn Oil Using Ethanol and Hexane

Batch extractions were performed to extract corn oil from ground corn, with a solvent-solid ratio of (a) 4 mL: 1 g, (b) 3 mL: 1 g, and (c) 2 mL: 1 g using ethanol and hexane as the solvents in a sealed glass vessel on a hot plate using a magnetic stir bar and a temperature probe. The glass vessel was purged with nitrogen prior to the extraction. The oil was extracted at 55° C. after 90 minutes of stirring at a speed of 350 rpm. The extractant was then membrane-filtered to obtain a clear solution of corn oil and the solvent, free of solid residues. The resulting extract was then placed in a vacuum oven at 100° C. with a trap at (−55° C.) to evaporate the solvent to yield the mass of the extracted corn oil.

### Extraction of Corn Oil Using Pressurized Propane for Solvent-Solid Ratio of 4:1

A Parr reactor (Stainless Steel, 160 mL, 2000 psi, 350° C.) was used to extract corn oil using pressurized propane. An external reservoir (Stainless Steel, 80 mL) was used for delivering propane into the reactor vessel. The reservoir, reactor, and the lines were initially purged with nitrogen to remove any air or moisture present. Propane was then admitted from an external tank into the storage reservoir immersed in dry ice for 15-20 minutes to condense the gas into liquid. Approximately 36 g of propane was loaded into the reservoir at room temperature. The propane in the reservoir was then pushed into the Parr reactor vessel using nitrogen gas at 2 MPa. A measured amount of ground corn was previously loaded into the reactor vessel such that the solvent-solid ratio was 4 mL: 1 g. The reactor vessel was heated to 55° C., stirring at 350 rpm. The pressure increased from 20 bar to 39 bar (20 bar of nitrogen gas and 19 bar of propane), maintaining a liquid propane (73.04 mL) phase in the reactor vessel. A reactor effluent line was connected to a back-pressure regulator (Zaiput Flow Technologies, Waltham, MA, USA) and operated at a pressure of 7-10 bar so that the propane remained in a liquid state at room temperature. The line from the back-pressure regulator was connected to a glass bottle containing hexane (15-20 mL) to capture the extractant oil as gaseous propane was bubbled through hexane following a batch extraction run. The resulting oil-hexane extractant was then placed in a vacuum oven at 100° C. with a trap at (−55° C.) to evaporate the solvent and recover corn kernel oil.

The system and process flow diagram for the extraction of corn kernel oil with (a) pressurized propane (pressure vessel with agitator) and (b) alternate liquid solvents is shown in FIG. 2. Similar designs for extractors and flash separators for recovering the extracted oil were used for both cases. As shown in FIG. 2, gaseous propane was fed to a single-stage compressor, where propane was compressed to a pressure of 20.1 bar, making the temperature reach 87° C. The vapor propane was then fed to the condenser to cool the propane down to 55° C. The condenser accounted for a low-pressure drop of 0.1 bar. The pressurized propane (1) was then fed to the extractor (pressure vessel with agitator in FIG. 2). Alternatively, for the ethanol and hexane models, liquid solvent was pumped to a pressure of 2 bar and fed to a heat exchanger at room temperature and heated to 55° C. and 2 bar. The liquid solvent (2) was then delivered to the extractor. The dashed lines of FIG. 2 represent alternatives, i.e. in one model propane was fed to the extractor (1), in another model ethanol was fed (2), and in a third model, hexane was fed (2) to the extractor. The extraction temperature was chosen to give an optimal yield of corn oil. A pneumatic conveyor used air/vacuum to convey finely grounded corn material (3) into the extractor. The extraction was carried out at 55° C., 20 bar, and 90 minutes, for propane, and at 55° C., 1-2 bar, and 90 minutes for ethanol and hexane. The total pressure comprised contributions from both nitrogen and propane. Following this extraction, the oil-laden mixture (5) was transferred from the extractor into the flash separator. The flash separator was operated under conditions such that the solvent was vaporized and left at the top while the liquid phase, corn oil, exited at the bottom (8). For the pressurized propane model, the flashed vapor at −22° C. and 1.1 bar was fed back (6) to the compressor to repeat the process. For the ethanol/hexane models, the solvent was heated up to a temperature of 67-90° C. to vaporize and then cooled down in a condenser to 55° C. and directly fed (7) to the extractor to repeat the process. The streams (6) and (7) represent alternative models depending on the solvent used. Similar to propane, they underwent negligible pressure drop across the condenser. The residual corn (4), now devoid of the corn oil, was used for ethanol production (see 102 of FIG. 1).

### Experimental Evaluation of Solvent Performance

Table 1 summarizes the results of the batch process for corn oil extraction using ethanol, hexane, and propane as solvents at different solvent-solid ratios for 90 min at 55° C. As a control, a known weight of the corn was vacuum-dried to obtain the moisture content, which was 0.13 g/g wet corn using Equation 1.

$$Moisure content ( \frac{g}{g} wet corn ) = \frac{weight of corn before drying - weight of corn after drying}{weight of corn before drying} \qquad ( Equation 1 )$$

$$Yield of oil ( \% ) = ( \frac{total amount of oil extracted ( g )}{total amount of oil corn used ( g )} ) * 100 \qquad ( Equation 2 )$$

The yield of the oil was measured using Equation 2. At the solvent/solid volume-to-mass ratio of 4:1, the oil yield on a dry corn basis using ethanol as the solvent was determined to be 4.8±0.3% using a glass vessel. The concentration of the extractant was 0.0132±0.003 g of oil/g of solvent. Table 1 represents the results for other solvent-solid ratios.

Hexane extraction was studied in a Parr steel reactor. The corn oil yield from the Parr reactor was 4.69% of the ground corn mass for a solvent-solid ratio of 4:1. The concentration of the extractant oils was estimated to be 0.04 g/g.

With pressurized propane as the solvent at the same solvent volume to mass of corn ratio (4:1), the oil yield on a dry basis was determined to be 2.22% of the ground corn mass, and the concentration of the corn oil in the extract was around 0.022 g/ml. Notably, the propane density was 0.493 g/ml, and the concentration of the corn oil could also be expressed as 0.02 g oil/g propane. For comparison, the corresponding value for ethanol was 0.01 g oil/g ethanol. The data demonstrate the utility of propane for extracting corn oil from corn kernels.

TABLE 1
Yield of oil obtained from various solvent-
extracted oils at different ratios at 55° C.

| | | Weight | | | | |
|---|---|---|---|---|---|---|
| | | of | Volume | Yield of | Yield of | Concen- |
| | | ground | of the | oil on a | oil on a | tration |
| | Solvent- | corn | solvent | dry | wet | (g of |
| | solid ratio | used | used | basis | basis | oil/g of |
| Solvent | (ml:g) | (g) | (ml) | (%) | (%) | solvent) |
| Ethanol | 4:1 | 4 | 16 | 4.80 | 4.18 | 0.013 |
| | 3:1 | 8 | 24 | 5.15 | 4.48 | 0.018 |
| | 2:1 | 4 | 8 | 1.46 | 1.27 | 0.008 |
| Hexane - | 4:1 | 10 | 40 | 4.69 | 4.08 | 0.016 |
| in Parr | 3:1 | 4.04 | 12 | 3.63 | 3.16 | 0.016 |
| reactor | 2:1 | 4 | 8 | 3.46 | 3.01 | 0.023 |
| Propane | 4:1 | 20.80 | 81.24 | 2.55 | 2.22 | 0.022 |
| (32 bar) | 3:1 | 17.97 | 53.85 | 2.50 | 2.18 | 0.022 |
| | 2:1 | 24.77 | 49.45 | 2.72 | 2.36 | 0.024 |

This Example investigated the yields of extracted corn oil from ground corn kernel using ethanol, hexane, and pressurized propane as the extraction solvent. Results showed that pressurized propane may be used as a food grade and efficient solvent for corn oil extraction. Non-limiting advantages associated with using pressurized propane include tuning the densities and transport properties for maximizing extraction, and the ease of separation of the propane from the extracted oil by simple pressure reduction with virtually no solvent traces in the oil.

### Example 2

This Example used post-extraction corn (according to Example 1) for ethanol fermentation. The fermentation process was carried out using a jacketed vessel filled with insulation and connected to a temperature control. To each sample (20 g), 70 ml of hot distilled water (90.6° C.) and 100 μL of α-amylase enzyme solution were added. The mixture was stirred at 83-85° C. for 90 minutes. The corn mash was then allowed to cool down overnight to ambient temperature. The following day, 200 mg of Urea, 2 g of dry yeast, and 0.5 mL of Glucoamylase enzyme solution were added to the mash. The mixture in the glass vessel containing the nutrients and the corn mash was placed in the insulation vessel at a controlled temperature of 32° C. The fermentation took place for 72 hours. After fermentation, the samples were centrifuged to separate water and ethanol from the corn mash. All the samples were subsequently analyzed using 1H NMR.

The ¹H NMR of the liquid sample obtained after fermentation was acquired using a 500 MHz Bruker A VIIIHD spectrometer. The NMR samples were run at operating conditions of 55° C., with 32 scans and a relaxation delay of 15 seconds. To identify the chemical shift (ppm) for methyl groups in ethanol, a reference sample with just standard ethanol was initially run, and the internal reference (DMSO) was dissolved in 600 μL of deuterated chloroform. After identification, all fermented samples (0.02-0.06 g) were added to 50 μL of DMSO and 600 μL of deuterated chloroform in NMR tubes for sample analysis. The number of millimoles of ethanol and the yield of ethanol (volume of ethanol/weight of the sample) % were calculated using the equations below:

$$Number of mmols of ethanol = \frac{( Area of methyl group in ethanol ) * ( Number of mmols of Int . Std . used ) * ( \frac{Number of protons in int . std .}{Number of protons in methyl group} )}{( Area of Internal Standard )}$$

$$Ethanol Yield ( \frac{v}{w} \% ) = \frac{weight of ethanol in NMR sample ( g )}{density of ethanol ( \frac{g}{mol} ) * weight of the total sample ( g )} * 100$$

### Study of ¹H NMR for Oil Samples

The ¹H NMR of commercially available corn oil and the corn oil samples extracted from ground corn using ethanol, hexane, and pressurized propane as a solvent (according to Example 1) were acquired using a 500 MHz Bruker A VIIIHD spectrometer. The NMR samples were run at operating conditions of 55° C., with 32 scans, and a relaxation delay of 15 seconds. Prior to the analysis, the samples were evaporated to remove the solvent. Approximately 0.02-0.3 g was dissolved in 600 μL of deuterated chloroform in NMR tubes for sample analysis. The components in the oil were identified with the help of a previous study. The estimation of vitamins in each extracted oil through the solvent and from the commercial oil was estimated as area % using the equation below:

$$Area ( \% ) = \frac{Area of Tocopherol identified}{Area of total components identified in NMR} * 100$$

### ¹H NMR Analysis of Commercial and Extracted Oils

Regions from 0 to 5 ppm should contain signals from triglycerides, monoglycerides, and diglycerides. Based on a previous study, the chemical shift of alpha-tocopherols (Vitamin E) was identified at 2.16 ppm. After confirming the presence of a compound at 2.16 ppm using reference peak tables in NMR, the area percentage of this vitamin E was estimated using the above equation. The area percent was low, as only 1% of the vitamins were identified in the oil. Table 2 shows the area percent for tocopherols in commercial corn oil and corn oil obtained via extraction using ethanol and pressurized propane (according to Example 1). Notably, the area percent of ethanol and propane extracted was 5 times higher than that of commercial corn oil. This indicates that the extraction process was effective at recovering the vitamins from the ground corn which are highly valuable in human diets, even when the propane yield is lower than the ethanol yield at 55° C.

TABLE 2
Area % for tocopherols in each extraction type of corn oil.

| Type | Area % for tocopherols |
|---|---|
| Commercial Corn Oil (CO) | 0.005 |
| Corn oil extracted using ethanol (EO) | 0.026 |
| Corn oil extracted using propane (PO) | 0.024 |

Additional experiments were conducted involving the fermentation of corn kernels to ethanol using corn kernels subjected to extraction either using ethanol, hexane, or propane. The results showing the ethanol yield are shown in Table 3, below.

TABLE 3
Fermentation of Corn Kernels (Post-Extraction) to Ethanol.

| Solvent used for extraction | (w/w) % of ethanol | (vol/w) % of ethanol |
|---|---|---|
| prior to fermentation | in product stream | in product stream |
| Control (no corn oil | 9.13 | 11.56 |
| extraction) | | |
| Ethanol (preliminary) | 7.22 | 9.14 |
| Hexane | 8.63 | 10.93 |
| Propane | 8.97 | 11.35 |

### Example 3

### Introduction

This Example investigates the integration of a pressurized propane-based corn kernel oil extraction step into the dry milling process, allowing the remaining solids to be utilized for ethanol fermentation. Comparative extractions using ethanol, hexane, and propane were conducted on ground corn at a solvent-to-solid ratio of 4 mL/g. Oil yields were 98.2±0.09% for ethanol, 94.2±0.01% for hexane, and 76.86±0.14% for pressurized propane.

### Materials and Methods

### Materials

Ground corn kernels were obtained from Trenton Agri Products LLC, Kansas, USA. Ethyl alcohol (pure, 200 proof, anhydrous, >99.5), n-hexane (99%), propane (98%) gas cylinder, ∝-amylase solution from Aspergillus Oryzae, yeast from Saccharomyces cerevisiae, amyloglucosidase solution from Aspergillus Niger, dimethyl sulfoxide, chloroform-d (99.95% deuteration degree), D-(−)-fructose (>99%), D-(+)-glucose (>99.5%), and D-(+)-maltose monohydrate (>99%) were purchased from Sigma Aldrich, USA. Urea (98+%) and D-(+)-sucrose (99.7%) were purchased from Thermo Fisher Scientific, USA. Commercial corn oil used in food consumption was purchased from Walmart, USA.

### Experimental Methods

### Extraction of Corn Oil Using Ethanol and Hexane

Batch extractions were conducted to extract corn oil from ground corn at solvent-to-solid ratios of (a) 4 mL/g, (b) 3 mL/g, and (c) 2 mL/g using ethanol and hexane as the solvents in a Parr reactor vessel (stainless steel, 160 mL, MAWP 13.79 MPa at 623.15 K). Prior to the extraction, the reactor vessel was purged with nitrogen to prevent oxidation. The extraction was carried out at 328.15 K for 90 minutes under continuous stirring at a speed of 350 rpm. Following extraction, the slurry was filtered using a membrane filter to obtain a clear solution of corn oil and the solvent, free of solid residues. The resulting filtrate was then placed in a vacuum oven at 373.15 K with a cold trap maintained at −328.15 K to evaporate the solvent to yield the mass of the extracted corn oil.

### Extraction of Corn Oil Using Pressurized Propane for a Solvent-to-Solid Ratio of 4:1

A Parr reactor (stainless steel, 160 mL) was used to extract corn oil using pressurized propane. An external reservoir (stainless steel, 80 mL) was used for delivering propane into the reactor vessel. Prior to extraction, the reservoir, reactor vessel, and lines were purged with nitrogen to remove residual air or moisture. Propane was then admitted from an external storage tank into the reservoir, which was immersed in dry ice for 25-30 minutes to condense the propane gas into liquid form. Approximately 24-40 g of liquid propane was loaded into the reservoir. A measured amount of ground corn was pre-loaded into the reactor vessel to achieve the desired solvent-to-solid ratio. The reactor vessel was purged with N₂. The reactor vessel was initially cooled using dry ice to facilitate the complete transfer of the liquid propane from the reservoir into the reactor. Propane transfer was facilitated by applying nitrogen gas at 2 MPa to the reservoir. The reactor vessel was then heated to 328.15 K and stirred at 350 rpm. As the temperature increased, the system pressure increased from approximately 20 bar (between −281.15 K and −278.15 K) to 3.6 MPa (at 328.15 K), maintaining a liquid propane phase (49-81 mL) within the reactor. The total pressure comprised contributions from both nitrogen and propane. Following extraction, the reactor effluent line was directed through a back-pressure regulator (Zaiput Flow Technologies, Waltham, MA, USA) and maintained at a pressure of 0.7-1.0 MPa to retain propane in its liquid state at room temperature. The effluent stream was bubbled through hexane (15-20 mL) contained in a glass vessel to capture the extracted oil as propane vaporized. The resulting oil-hexane mixture was then placed in a vacuum oven at 373.15 K with a cold trap maintained at −328.15 K to evaporate hexane and recover the extracted corn kernel oil.

### Fermentation of Extracted Corn to Ethanol

The solid corn residue remaining after oil extraction using ethanol, hexane, and pressurized propane was utilized for ethanol fermentation. The fermentation process was conducted in a glass vessel sealed with a rubber stopper and fitted with an open syringe to allow CO₂ release. The process consisted of three sequential steps: liquefaction, saccharification, and fermentation. In the liquefaction step, 20-100 g of extracted corn residue was added to 70-350 mL of hot water and was stirred for 15 minutes. During the saccharification step, 100 μl of ∝-amylase solution was added to the corn slurry and was stirred for 90 minutes while maintaining the temperature between 348.15-358.15 K. After 90 minutes, the mash was cooled down to room temperature and stored overnight at 275.85 K. Later, during the fermentation step, 0.2 g of urea, 2 g of dry yeast, and 0.5 ml of glucoamylase solution were added to 20-100 g of corn mash. Fermentation was carried out at 305.15 K and took up to 72 h to complete. Following fermentation, the samples were centrifuged to separate the liquid phase (containing water and ethanol) from the residual solids. The ethanol content in the supernatant phase was quantified using NMR spectroscopy.

### ¹H NMR Analysis of Fermentation Samples

The ¹H NMR of the liquid samples obtained after fermentation of corn residue (following oil extraction with ethanol, hexane, and pressurized propane) was acquired using a 500 MHz Bruker AVIIIHD spectrometer. The NMR measurements were performed under operating conditions of 328.15 K, with 32 scans and a relaxation delay of 15 seconds. To identify the characteristic chemical shift (ppm) of ethanol methyl protons, a reference sample containing standard ethanol and an internal standard (dimethyl sulfoxide, DMSO) was prepared in 600 μL of deuterated chloroform (CDCl₃). Subsequently, fermented samples (0.02-0.06 g) were prepared by adding 50 μL of DMSO and 600 μL of CDCl₃ to NMR tubes. The methyl proton signal of ethanol was integrated relative to the DMSO peak to quantify ethanol concentration. The amount of ethanol (mmol) and ethanol titer (% v/w, defined as the volume of ethanol/weight of the sample) were calculated using Equations 3 and 4, respectively.

$${N}_{E} = ( \frac{{A}_{C 1 E} {N}_{IS} ( \frac{{HI}_{IS}}{{HI}_{C 1 E}} )}{{A}_{IS}} ) \qquad ( Equation 3 )$$

N_{E}: mmol of ethanol, N_{IS}: mmol of internal standard, A_{C1E}: area of methyl resonance for ethanol, A_{IS}: area of internal standard resonance, HI_{C1E}: number of protons associated with methyl resonance for ethanol, HI_{IS}: number of protons associated with internal standard resonance.

$$Ethanol Titer ( \frac{v}{w} \% ) = \frac{{N}_{E} ( mol ) * MW ( \frac{g}{mol} )}{ρ ( \frac{g}{mol} ) * W ( g )} * 100 \qquad ( Equation 4 )$$

N_{E}: mols of ethanol produced, MW: molecular weight of ethanol, p: density of ethanol, W: weight of the sample analyzed.

### ¹H NMR Analysis of Oil Samples

¹H NMR spectroscopy was used to analyze commercially available corn oil and corn oil samples extracted from ground corn using ethanol and pressurized propane as solvents in a 500 MHz Bruker A VIIIHD spectrometer. The NMR measurements were performed at 328.15 K, with 32 scans and a relaxation delay of 15 seconds. Prior to the analysis, all oil samples were evaporated to remove residual solvents. Approximately, 0.02-0.3 g of corn oil was dissolved in 600 μL of deuterated chloroform (CDCl₃) and transferred to NMR tubes for analysis. The oil components were identified based on the characteristic chemical shifts (ppm). In particular, the signal corresponding to tocopherols (Vitamin E), an antioxidant compound present naturally in corn oil, was observed at 2.15-2.16 ppm. Tocopherol content in the oil samples was characterized based on the area percentage of the corresponding peak, using Equation 5.

$$TI = \frac{{A}_{tocoph}}{{A}_{sum corn oil}} \qquad ( Equation 5 )$$

TI: tocopherol index, A_{tocoph}: area of resonance for tocopherol 2.15-2.16 ppm, A_{Sum corn oil}: the sum of the area of all resonances attributed to corn oil.

### HPLC Analysis of Extracted Oils

High performance liquid chromatography (HPLC) analysis of residual sugar and water solubles was performed using an Agilent 1100 (Variable Wavelength Detector, Quaternary Pump, USA), equipped with an automatic sample injector, a refractive index detector (RID), and a Biorad Aminex HPX-87H column with an appropriate guard column. The mobile phase consisted of HPLC grade water (0.2 μm filtered and degassed) at a flowrate of 0.4 mL/min, an injection volume of 10-50 μL, a column temperature of 353.15 K, and an RID temperature of 328.15 K. Standard solutions of sugars found in corn oil-fructose, sucrose, maltose, and glucose-were prepared at concentrations ranging from 10-70 mM in distilled water for calibration. The retention times for the sugars were observed at 15 min (fructose), 14 min (glucose), 11.5 min (maltose), and 33.82 min (sucrose), with additional peaks found for sucrose. Oil samples extracted using ethanol, hexane, and pressurized propane were treated with distilled water at a weight ratio of 3:1 (water to solvent-oil mixture) to extract soluble sugars. The water extraction of the oil-ethanol mixture was complicated due to ethanol's tendency to partition between phases. However, the 3:1 dilution minimized any potential interference with the HPLC column. The aqueous phase was collected and analyzed by HPLC, and total sugar concentrations were determined using calibration curves generated from the standard sugar solutions.

### Results and Discussion

### Experimental Evaluation of Solvent Performance

FIG. 3 showcases the results of batch corn oil extraction using ethanol, hexane, and pressurized propane at different solvent-solid ratios, conducted for 90 min at 328.15 K. The moisture content of the ground corn was determined by vacuum drying and was measured as 0.13 g water/g wet corn based on Equation 1 (Example 1, above). The oil yield was calculated using Equation 6 (note the different definition of oil yield as compared to Equation 2 in Example 1, above).

$$Yield of oil ( \% ) = ( \frac{total amount of oil extracted ( g )}{total amount of oil present in corn ( g )} ) * 100 \qquad ( Equation 6 )$$

At a solvent-solid ratio of 4 mL/g, the oil yield on a dry corn basis using ethanol was 98.24±0.09%, with the concentration of the extractant being 0.011±0.003 g/mL. For both ethanol and hexane-based extractions, decreasing solvent-to-solid ratios resulted in reduced oil yields. However, even at the lowest solvent-solid ratios studied, extending the extraction duration to 7-10 h at 328.15 K enabled complete oil recovery (100% yield). These results indicate that ethanol and hexane-based extractions are limited by mass transfer.

Using pressurized propane at a solvent-to-solid ratio of 4 mL/g, the oil yield on a dry corn basis was 76.86±0.14%, with an extractant concentration of 0.008±0.001 g/mL. Based on the density of liquid propane (0.493 g/ml) at the operating conditions, the oil extraction normalized with solvent weight corresponds to 0.02 g oil/g propane, compared to 0.01 g oil/g ethanol. These data demonstrate that by adjusting the pressure and temperature of the pressurized propane, its solvating power can be tuned to enhance corn oil extraction from the corn kernels.

### Tocopherol Content in Corn Oil Extracts

FIG. 4 shows the area percentage of tocopherols (Vitamin E) detected in commercial corn oils and corn oils extracted using pressurized propane and ethanol. The commercial corn exhibited the lowest tocopherol fraction percentage of 0.003, while propane and ethanol-extracted oils showed higher tocopherol fraction percentages of 0.010 and 0.013, respectively. The low tocopherol content observed in commercial corn oil is likely due to losses incurred during the wet milling process, which includes multiple refining steps such as bleaching and steam deodorization. Notably, steam deodorization is known to degrade tocopherols, contributing to their reduced presence in the final oil product. In contrast, the absence of the refining steps combined with the higher solubility of tocopherols in organic solvents and hydrocarbons explains their higher recovery in oils extracted using pressurized propane and ethanol.

Ethanol Yield from Fermentation of Extracted Corn Residue

The standard ethanol spectrum obtained from 1H NMR analysis was compared to the spectra of fermented ethanol samples derived from corn residues following oil extraction with ethanol, hexane, and pressurized propane. As shown in FIG. 5, an ethanol titer of 11.6 (v/w %) was achieved from the fermentation of commercial corn (without prior oil extraction). Among the extracted corn residues, propane-extracted corn residue exhibited the highest ethanol titer of 11.36 v/w %, followed by hexane-extracted corn residue at 10.93 v/w % and ethanol-extracted corn residue at 9.2 v/w %. The reduction in ethanol titer from solvent-extracted corn residues relative to commercial corn may be attributed to the co-extraction of sugars during the oil extraction process. Solvents used for oil extraction may extract not only lipids but also soluble sugars, thereby reducing the fermentable sugar content remaining in the corn residue for ethanol fermentation (see “Sugar Co-Extraction Analysis in Oil Samples” section, below).

### Sugar Co-Extraction Analysis in Oil Samples

HPLC analysis was performed on oil samples extracted using ethanol, hexane, and pressurized propane to evaluate the potential co-extraction of sugars during the extraction process. Calibration curves for the assessed sugars (fructose, maltose, and glucose) were obtained. Analysis of the ethanol-extracted oil samples revealed peaks corresponding to both fructose and sucrose. The quantified fructose content corresponded to approximately 6.8% of the mass of the corn used. Fructose peaks were also detected in propane and hexane-based extracted oil samples, but with significantly smaller peak areas compared to the area associated with ethanol-extracted corn oil. Quantification of sucrose in ethanol-based extracted oil samples was challenging due to the appearance of multiple peaks associated with sucrose isomers in the HPLC chromatogram, requiring calibration for all the isomers. Despite this, the presence of both fructose and sucrose in ethanol-based extracted oil samples provides supporting evidence for the reduced ethanol yields observed during the fermentation of ethanol-extracted corn residue, compared to propane and hexane-extracted corn residues.

### CONCLUSIONS

This Example investigated the extraction of corn oil from ground corn kernel using ethanol, hexane, and pressurized propane as solvents. The results demonstrated that pressurized propane may be used as food-grade solvent for corn oil extraction, offering several non-limiting advantages over conventional solvents. Notably, propane allowed for efficient oil recovery and simple solvent separation by depressurization, yielding virtually solvent-free oil. Propane-based extraction also preserved more fermentable sugars in the residual corn solids, leading to higher ethanol titer during fermentation compared to residues from ethanol and hexane-based extractions. This highlights propane's potential for integrated biorefinery applications where both oil and ethanol are co-produced.

Additional information relating to the present disclosure may be found in U.S. provisional patent application No. 63/712,616, filed Oct. 28, 2024, the entire contents of which are incorporated herein by reference.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

If not already included, all numeric values of parameters in the present disclosure are proceeded by the term “about” which means approximately. This encompasses those variations inherent to the measurement of the relevant parameter as understood by those of ordinary skill in the art. This also encompasses the exact value of the disclosed numeric value and values that round to the disclosed numeric value.

In recognition of the inherent nature of chemical synthesis, throughout the present disclosure, terms and phrases such as “absence,” “free,” “does not comprise,” etc. encompass, but do not require a perfect absence of the referenced entity.

The term “type” as used herein refers to chemical formula such that a single type means the same chemical formula and different type means different chemical formula. Similarly, use of “more” as in “one or more” refers to use of different types of the relevant entity.

Terms such as “comprising” and the like may be replaced with terms such as “consisting” and the like.

What is claimed is:

1. A method for processing corn kernels, the method comprising:
(a) exposing corn kernels to pressurized propane to extract corn oil from the corn kernels in a stirred tank extractor comprising an impeller, thereby providing an extraction mixture comprising extracted corn oil, pressurized propane, and processed corn kernels; and
(b) separating the extracted corn oil from the extraction mixture, thereby providing separated corn oil.

2. The method of claim 1, wherein the separating step (b) comprises evaporating propane from the extraction mixture to provide a vapor propane stream.

3. The method of claim 2, further comprising liquifying the vapor propane stream to provide re-liquified propane and using the re-liquified propane in an additional exposing step (a).

4. The method of claim 1, wherein the stirred tank extractor is a continuous stirred tank extractor.

5. The method of claim 1, wherein the exposing step (a) is carried out at an extraction pressure in a range of from 7 to 150 bar; an extraction temperature in a range of from 20 to 100° C.; an extraction time in a range of from 15 to 360 min; a solvent-to-solid ratio in a range of from 10:1 to 1:1; and a mixing rate of from 10 rpm to 1000 rpm.

6. The method of claim 5, wherein the extraction pressure is in a range of from 20 to 40 bar; the extraction temperature is in a range of from 50 to 60° C.; the extraction time is in a range of from 60 to 100 min; the solvent-to-solid ratio is in a range of from 5:1 to 3:1; and the mixing rate of from 250 rpm to 500 rpm.

7. The method of claim 1, wherein the separating step (b) is carried out by feeding a liquid phase portion of the extraction mixture, the liquid phase portion comprising the extracted corn oil and liquid propane, to a separator configured to contain the liquid phase portion and evaporate the liquid propane to provide a vapor propane stream.

8. The method of claim 7, wherein the separating step (b) is carried out at a separation temperature in a range of from −30 to 55° C. and separation pressure from 1 to 70 bar.

9. The method of claim 1, wherein the separated corn oil is suitable for human consumption.

10. The method of claim 1, wherein the separated corn oil has a tocopherol fraction of at least at least 0.0075%.

11. The method of claim 1, wherein the method provides a corn oil yield of at least 70%.

12. The method of claim 1, wherein the separated corn oil has a tocopherol fraction of at least at least 0.0075% and the method provides a corn oil yield of at least 70%.

13. The method of claim 1, further comprising subjecting the processed corn kernels to a fermentation step in an ethanol plant.

14. The method of claim 13, wherein the fermentation step results in an ethanol titer of at least 11 (v/w) %.

15. The method of claim 1, wherein the corn kernels are ground corn kernels from an ethanol plant and the method further comprises subjecting the processed corn kernels to a fermentation step in the ethanol plant.

16. The method of claim 1, wherein the pressurized propane has a purity of not more than 98%.

17. The method of claim 1, wherein the separating step (b) provides a vapor propane stream and the method further comprises compressing the vapor propane stream to a liquid propane stream while transferring heat released during the compressing for use in another step of the method.

18. The method of claim 17, wherein the another step of the method is heating the corn kernels before the exposing step (a), during the exposing step (a), or both.

19. The method of claim 18, wherein the exposing step (a) is carried out at an extraction pressure in a range of from 20 to 40 bar; an extraction temperature is in a range of from 50 to 60° C.; an extraction time is in a range of from 60 to 100 min; a solvent-to-solid ratio is in a range of from 5:1 to 3:1; and a mixing rate of from 250 rpm to 500 rpm.

20. The method of claim 19, wherein the method provides a corn oil yield of at least 70%.
